(19) United States
(12) Patent Application Publication
(10) Pub. No.: US 2013/0233721 A1
(43) Pub. Date: Sep. 12, 2013

(54) PROCESS FOR PREPARING AMINO HYDROCARBONS BY DIRECT AMINATION OF HYDROCARBONS

(71) Applicant: BASF SE, Ludwigshafen (DE)

(72) Inventors: Bernd Bastian SCHAACK, Ludwigshafen (DE); Alexander Panchenko, Ludwigshafen (DE); Philipp Brueggemann, Ludwigshafen (DE)

(73) Assignee: BASF SE, Ludwigshafen (DE)

(21) Appl. No.: 13/785,980

(22) Filed: Mar. 5, 2013

Related U.S. Application Data
(60) Provisional application No. 61/607,007, filed on Mar. 6, 2012.

(51) Int. Cl. C07C 209/02; C25B 3/04

(52) U.S. Cl. CPC C07C 209/02; C25B 3/04
USPC 205/431

(57) ABSTRACT

The present invention relates to a process for direct amination of hydrocarbons to amino hydrocarbons, comprising
(a) the reaction of a reactant stream E comprising at least one hydrocarbon and at least one aminating reagent to give a reaction mixture R comprising at least one amino hydrocarbon and hydrogen in a reaction zone RZ, and
(b) electrochemical removal of at least a portion of the hydrogen formed in the reaction from the reaction mixture R by means of at least one gas-tight membrane electrode assembly which is in contact with the reaction zone RZ on the retentate side and which has at least one selectively proton-conducting membrane,
at least a portion of the hydrogen being oxidized over an anode catalyst to protons on the retentate side of the membrane, and the protons, after passing through the membrane, being partly or fully reduced by applying a voltage over a cathode catalyst to give hydrogen on the permeate side.

The present invention relates to a process for direct amination of hydrocarbons to amino hydrocarbons, comprising

(a) the reaction of a reactant stream E comprising at least one hydrocarbon and at least one aminating reagent to give a reaction mixture R comprising at least one amino hydrocarbon and hydrogen in a reaction zone RZ, and

(b) electrochemical removal of at least a portion of the hydrogen formed in the reaction from the reaction mixture R by means of at least one gas-tight membrane electrode assembly which is in contact with the reaction zone RZ on the retentate side and which has at least one selectively proton-conducting membrane,

at least a portion of the hydrogen being oxidized over an anode catalyst to protons on the retentate side of the membrane, and the protons, after passing through the membrane, being partly or fully reduced by applying a voltage over a cathode catalyst to give hydrogen on the permeate side.

In general, the commercial synthesis of amino hydrocarbons typically proceeds via multistage synthesis routes. One example which can be cited is the preparation of aniline from benzene: this involves first using benzene to prepare a benzene derivative such as nitrobenzene, chlorobenzene or phenol. This is subsequently converted to aniline in one-stage or multistage reactions.

However, there are also known processes for direct preparation of amino hydrocarbons from the corresponding hydrocarbons. This so-called direct amination (of benzene to aniline) was described for the first time by Wibaut (Berichte 1917, 50, 541-546). In this case, the reaction of the hydrocarbon with an aminating reagent to give the corresponding amino hydrocarbon takes place with release of hydrogen. However, reactions of this type are generally limited by the position of the thermodynamic equilibrium. In the case of direct amination of benzene, the equilibrium conversion at typical reaction temperatures of 350° C. is less than 0.5 mol %.

In order to be able to conduct the direct amination in an economically viable manner, a shift in the position of the thermodynamic equilibrium to the side of the products (amino hydrocarbons and hydrogen) is necessary. One way of achieving this is by removal of the hydrogen formed from the reaction zone, which is described in various processes:

WO 2007/096297 and WO 2000/69804 describe a process for direct amination of aromatic hydrocarbons to the corresponding amino hydrocarbons, wherein the hydrogen formed is removed from the reaction mixture by oxidation over reducible metal oxides. These processes have the disadvantage that the reducible metal oxides have to be regenerated again with oxygen after a certain time. This means costly interruptions of the process, since the direct amination of the hydrocarbons and the regeneration of the reducible metal oxides typically do not proceed under the same conditions. For regeneration of the catalyst, the reactor must therefore typically be decompressed, purged and inertized.

A further unwanted side reaction which occurs in the direct amination of hydrocarbons to amino hydrocarbons is the decomposition of ammonia to hydrogen. This decomposition is disadvantageous since, firstly, the ammonia reactant is lost and, secondly, the hydrogen formed in the decomposition leads to a further unfavorable shift in the equilibrium position in the direction of the reactants. In the case of the catalysts described in WO 2007/096297 and WO 2000/69804, the unwanted decomposition of ammonia increases with rising degree of reduction of the metal oxides, such that the equilibrium position is shifted ever further in the direction of the reactants as the degree of reduction rises.

WO 2007/099028 describes a direct amination process of aromatic hydrocarbons to the corresponding amino hydrocarbons, wherein, in a first step, the heterogeneously catalyzed direct amination is performed and, in a second step, the hydrogen formed in the first step is converted by the reaction with an oxidizing agent such as air, oxygen, CO, CO₂, NO and/or N₂O. The use of oxidizing agents such as oxygen leads to the oxidation of ammonia and to the formation of further by-products. This leads to higher material costs and to additional workup steps, as a result of which the economic viability of the process is worsened.

WO 2008/009668 likewise describes a process for direct amination of aromatic hydrocarbons. The removal of the hydrogen from the reaction mixture is achieved here by performing the direct amination with addition of compounds which react with the hydrogen formed in the direct amination. Nitrobenzene and carbon monoxide, for example, are described as compounds added in the direct amination. In this process too, the above-described disadvantages occur.

WO 2007/025882 describes the direct amination of aromatic hydrocarbons to the corresponding amino hydrocarbons, wherein hydrogen is physically removed from the reaction mixture. The removal is effected here by a selectively hydrogen-pervious membrane, which means that hydrogen migrates through the membrane as the H₂ molecule. The membrane materials used are preferably palladium and palladium alloys. The diffusion rate in this process depends on the partial pressure difference of the hydrogen between the retentate side and permeate side of the membrane. In order to achieve higher diffusion rates, it is necessary to work at higher pressure differences, which place high demands on the mechanical stability of the membrane. In addition, the academic literature states that achievement of a sufficiently high diffusion rate requires temperatures above 300° C. (Top. Catal. 2008, 51, 107-122). Furthermore, appropriate apparatuses for compression and expansion of the gas mixture must be present for formation of the pressure differences. For thermodynamic reasons, moreover, a certain proportion of the hydrogen always remains in the retentate. This has an adverse effect on the position of the thermodynamic equilibrium.

WO 2011/003964, WO 2011/003932, WO 2011/003933 and WO 2011/003934 describe the direct amination of hydrocarbons with ex situ removal of the hydrogen formed. The local combination of reaction zone and hydrogen removal is not described in the details given, and this results in a higher apparatus complexity.

It is therefore an object of the present invention to provide a process for direct amination of hydrocarbons to amino hydrocarbons, wherein the hydrogen formed is removed with maximum efficacy from the reaction mixture, and which overcomes the above-described disadvantages of the processes known to date. In addition, the process shall enable a shift in the position of the thermodynamic equilibrium to the side of the products, and, more particularly, the hydrogen formed shall be removed directly from the reaction zone. In addition, the process shall be characterized by a very favorable energy balance, and a minimum apparatus complexity.

This object is achieved in accordance with the invention by a process for direct amination of hydrocarbons to amino hydrocarbons, comprising

(a) the reaction of a reactant stream E comprising at least one hydrocarbon and at least one aminating reagent to give a reaction mixture R comprising at least one amino hydrocarbon and hydrogen in a reaction zone RZ, and

(b) electrochemical removal of at least a portion of the hydrogen formed in the reaction from the reaction mixture R by means of at least one gas-tight membrane electrode assembly which is in contact with the reaction zone RZ on the retentate side and which has at least one selectively proton-conducting membrane,

at least a portion of the hydrogen being oxidized over an anode catalyst to protons on the retentate side of the membrane, and the protons, after passing through the membrane, being partly or fully reduced by applying a voltage over a cathode catalyst to give hydrogen on the permeate side.

A membrane electrode assembly (MEA) is understood in the context of the present invention to mean an electrochemical unit comprising at least one membrane having an anode side with an anode and a cathode side with a cathode. The cathodes and anodes of the present invention additionally have at least one cathode catalyst and anode catalyst respectively. A cathode catalyst is in contact with the cathode and is a catalyst. An anode catalyst is in contact with the anode and is a catalyst. The anode and anode catalyst may be one and the same material or may consist of different materials. Cathode and cathode catalyst may be one and the same material or may consist of different materials.

MEA which is in contact with the reaction zone RZ on the retentate side is understood in the context of the present invention to mean that the reaction zone RZ directly adjoins the retentate side of the MEA. As a result of this, the hydrogen is electrochemically removed from the reaction zone and hence from the reaction mixture R by means of the MEA. As a result of this, the reaction zone and the MEA form one unit in spatial and process technology terms, which leads to advantages in terms of process technology and construction. This should be distinguished from onward conduction of the reaction mixture R and later contacting with the MEA.

The membrane of the MEA has at least one electrode catalyst on each side, the electrode catalyst present on the retentate side being referred to in the context of this description as anode catalyst, and the electrode catalyst present on the permeate side as cathode catalyst.

Reaction zone RZ in the context of the present invention is understood to mean the region in which the chemical reaction of at least one hydrocarbon and at least one aminating reagent to give the reaction mixture R takes place.

Reactant stream E is understood in the context of the present invention to mean the stream which is conducted into the reaction zone and comprises at least one hydrocarbon and at least one aminating reagent.

In the reaction zone RZ, in accordance with the invention, the reaction according to step (a) takes place. In the same reaction zone RZ, in addition, at least a portion of the hydrogen formed in the reaction is removed electrochemically from the reaction mixture R by means of a gas-tight membrane electrode assembly (MEA).

The MEA comprises, in accordance with the invention, at least one selectively proton-conducting membrane and, on each side of the membrane, at least one electrode catalyst, at least a portion of the hydrogen being oxidized over the anode catalyst to protons on the retentate side of the membrane, and the protons, after passing through the membrane, reacting with an oxidizing agent over the cathode catalyst to give water, generating electrical power, on the permeate side.

In a preferred embodiment, the MEA can be combined with one or more gas diffusion layers (GDLs), which have the feature that, as well as improved gas diffusion, active components applied can accelerate the conversion of the reactant stream E; the same function can be fulfilled by the electrode catalyst on the retentate side of the MEA.

Compared to processes for direct amination of hydrocarbons known from the prior art, the invention described has the advantage that the synthesis of the amino hydrocarbons can be conducted in one stage and continuously, and hence inconvenient and costly production shutdowns are avoided. Moreover, the process described does not need any gaseous oxidizing agent, such as air, oxygen, CO, CO₂, NO or N₂O, in the reaction mixture R, as a result of which by-product formation can be avoided and costs can be saved. In addition, the electrochemical removal of the hydrogen is much more effective compared to removal by means of conventional hydrogen-selective membranes. This means that, for the same separation performance, the membrane area required can be reduced or, for the same membrane area, much more hydrogen can be removed. A particular advantage of the process according to the invention is therefore the spatial combination of reaction zone RZ and electrochemical removal of the hydrogen formed from the reaction mixture R. This allows more efficient removal of the hydrogen and hence an improved shift in the thermodynamic equilibrium to the side of the products, and also an apparatus simplification. This results in an economic improvement of the direct amination of hydrocarbons to amino hydrocarbons compared to known processes.

The invention is described in detail hereinafter. Combinations of preferred embodiments do not leave the scope of the present invention. This is especially true in relation to preferred embodiments of process steps (a) and (b), which can be combined with one another.

### Hydrocarbons

According to the invention, the reactant stream E comprises at least one hydrocarbon. Suitable hydrocarbons which can be used in the process according to the invention are, for example, hydrocarbons such as aromatic hydrocarbons, aliphatic hydrocarbons and cycloaliphatic hydrocarbons, which may have any substitution and may have heteroatoms and double or triple bonds within their chain or their ring/their rings. In the amination process according to the invention, preference is given to using aromatic hydrocarbons and heteroaromatic hydrocarbons.

Suitable aromatic hydrocarbons are, for example, unsaturated cyclic hydrocarbons which have one or more rings and comprise exclusively aromatic C-H bonds. Preferred aromatic hydrocarbons have one or more 5- and/or 6-membered rings.

A heteroaromatic hydrocarbon is understood to mean those aromatic hydrocarbons in which one or more of the carbon atoms of the aromatic ring is/are replaced by a heteroatom selected from N, O and S.

The aromatic hydrocarbons or the heteroaromatic hydrocarbons may be substituted or unsubstituted. A substituted aromatic or heteroaromatic hydrocarbon is understood to mean compounds in which one or more hydrogen atoms which is/are bonded to a carbon atom and/or heteroatom of the aromatic ring is/are replaced by another radical. Suitable radicals are, for example, substituted or unsubstituted alkyl, alkenyl, alkynyl, heteroalkyl, heteroalkenyl, heteroalkynyl, cycloalkyl and/or cycloalkynyl radicals; halogen, hydroxyl, alkoxy, aryloxy, amino, amido, thio and phosphino. Preferred radicals of the aromatic or heteroaromatic hydrocarbons are selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido, where C₁₋₆ relates to the number of carbon atoms in the main chain of the alkyl radical, of the alkenyl radical or of the alkynyl radical, and C₃₋₈ to the number of carbon atoms of the cycloalkyl or cycloalkenyl ring. It is also possible that the substituents (radicals) of the substituted aromatic or heteroaromatic hydrocarbon have further substituents in turn.

The number of substituents (radicals) of the aromatic or heteroaromatic hydrocarbon is arbitrary. In a preferred embodiment, the aromatic or heteroaromatic hydrocarbon has, however, at least one hydrogen atom which is bonded directly to a carbon atom or a heteroatom of the aromatic or heteroaromatic ring. Thus, a 6-membered ring preferably has 5 or fewer substituents (radicals) and a 5-membered ring preferably has 4 or fewer substituents (radicals). A 6-membered aromatic or heteroaromatic ring more preferably bears 4 or fewer substituents, even more preferably 3 or fewer substituents (radicals). A 5-membered aromatic or heteroaromatic ring preferably bears 3 or fewer substituents (radicals), more preferably 2 or fewer substituents (radicals).

In a particularly preferred embodiment of the process according to the invention, an aromatic or heteroaromatic hydrocarbon of the general formula

(A)−(B)ₙ

is used, where the symbols are each defined as follows:
A is independently aryl or heteroaryl, A is preferably selected from phenyl, diphenyl, benzyl, dibenzyl, naphthyl, anthracene, pyridyl and quinoline;
n is 0 to 5, preferably 0 to 4, especially in the case when A is a 6-membered aryl or heteroaryl ring; in the case that A is a 5-membered aryl or heteroaryl ring, n is preferably 0 to 4; irrespective of the ring size, n is more preferably 0 to 3, most preferably 0 to 2 and especially 0 to 1; the remaining hydrocarbon atoms or heteroatoms of A which do not bear any substituents B bear hydrogen atoms, or optionally no substituents;
B is independently selected from the group consisting of alkyl, alkenyl, alkynyl, substituted alkyl, substituted alkenyl, substituted alkynyl, heteroalkyl, substituted heteroalkyl, heteroalkenyl, substituted heteroalkenyl, heteroalkynyl, substituted heteroalkynyl, cycloalkyl, cycloalkenyl, substituted cycloalkyl, substituted cycloalkenyl, halogen, hydroxyl, alkoxy, aryloxy, carbonyl, amino, amido, thio and phosphino; B is preferably independently selected from C₁₋₆-alkyl, C₁₋₆-alkenyl, C₁₋₆-alkynyl, C₃₋₈-cycloalkyl, C₃₋₈-cycloalkenyl, alkoxy, aryloxy, amino and amido.

The term “independently” means that, when n is 2 or greater, the substituents B may be identical or different radicals from the groups mentioned.

In the present application, alkyl is understood to mean branched or unbranched, saturated acyclic hydrocarbyl radicals. The alkyl radicals used preferably have 1 to 20 carbon atoms, more preferably 1 to 6 carbon atoms and especially 1 to 4 carbon atoms. Examples of suitable alkyl radicals are methyl, ethyl, n-propyl, i-propyl, n-butyl, t-butyl and i-butyl.

In the present application, alkenyl is understood to mean branched or unbranched, acyclic hydrocarbyl radicals which have at least one carbon-carbon double bond. The alkenyl radicals have preferably 2 to 20 carbon atoms, more preferably 2 to 6 carbon atoms and especially 2 to 3 carbon atoms. Suitable alkenyl radicals are, for example, vinyl and 2-propenyl.

In the present application, alkynyl is understood to mean branched or unbranched, acyclic hydrocarbyl radicals which have at least one carbon-carbon triple bond. The alkynyl radicals preferably have 2 to 20 carbon atoms, more preferably 1 to 6 carbon atoms and especially 2 to 3 carbon atoms. Examples of suitable alkynyl radicals are ethynyl and 2-propynyl.

Substituted alkyl, substituted alkenyl and substituted alkynyl are understood to mean alkyl, alkenyl and alkynyl radicals in which one or more hydrogen atoms which are bonded to one carbon atom of these radicals are replaced by another group. Examples of such other groups are halogen, aryl, substituted aryl, cycloalkyl, cycloalkenyl, substituted cycloalkyl, substituted cycloalkenyl and combinations thereof. Examples of suitable substituted alkyl radicals are benzyl and trifluoromethyl.

The terms heteroalkyl, heteroalkenyl and heteroalkynyl are understood to mean alkyl, alkenyl and alkynyl radicals in which one or more of the carbon atoms in the carbon chain is replaced by a heteroatom selected from N, O and S. The bond between the heteroatom and a further carbon atom may be saturated or unsaturated.

In the present application, cycloalkyl is understood to mean saturated cyclic nonaromatic hydrocarbyl radicals formed from a single ring or a plurality of fused rings. The cycloalkyl radicals have preferably between 3 and 8 carbon atoms and more preferably between 3 and 6 carbon atoms. Suitable cycloalkyl radicals are, for example, cyclopropyl, cyclopentyl, cyclohexyl, cyclooctanyl and bicyclooctyl.

In the present application, cycloalkenyl is understood to mean partly unsaturated, cyclic nonaromatic hydrocarbyl radicals which have a single fused ring or a plurality of fused rings. The cycloalkenyl radicals have preferably 3 to 8 carbon atoms and more preferably 5 to 6 carbon atoms. Suitable cycloalkenyl radicals are, for example, cyclopentenyl, cyclohexenyl and cyclooctenyl.

Substituted cycloalkyl and substituted cycloalkenyl radicals are cycloalkyl and cycloalkenyl radicals, in which one or more hydrogen atoms of any carbon atom of the carbon ring is replaced by another group. Such other groups are, for example, halogen, alkyl, alkenyl, alkynyl, substituted alkyl, substituted alkenyl, substituted alkynyl, aryl, substituted aryl, cycloalkyl, cycloalkenyl, substituted cycloalkyl, substituted cycloalkenyl, an aliphatic heterocyclic radical, a substituted aliphatic heterocyclic radical, heteroaryl, substituted heteroaryl, alkoxy, aryloxy, boryl, phosphino, amino, silyl, thio, seleno and combinations thereof. Examples of substituted cycloalkyl and cycloalkenyl radicals are 4-dimethylamino-cyclohexyl and 4,5-dibromocyclohept-4-enyl.

In the context of the present application, aryl is understood to mean aromatic radicals which have a single aromatic ring or a plurality of aromatic rings which are fused, joined via a covalent bond or joined by a linking unit, such as a methylene or ethylene unit. Such linking units may also be carbonyl units, as in benzophenone, or oxygen units, as in diphenyl ether, or nitrogen units, as in diphenylamine. The aryl radicals preferably have 6 to 20 carbon atoms, more preferably 6 to 8 carbon atoms and especially preferably 6 carbon atoms. Examples of aromatic rings are phenyl, naphthyl, diphenyl, diphenyl ether, diphenylamine and benzophenone.

Substituted aryl radicals are aryl radicals in which one or more hydrogen atoms which are bonded to carbon atoms of the aryl radical are replaced by one or more groups such as alkyl, alkenyl, alkynyl, substituted alkyl, substituted alkenyl, substituted alkynyl, cycloalkyl, cycloalkenyl, substituted cycloalkyl, substituted cycloalkenyl, heterocyclo, substituted heterocyclo, halogen, halogen-substituted alkyl (e.g. CF₃), hydroxyl, amino, phosphino, alkoxy and thio. In addition, one or more hydrogen atoms bonded to carbon atoms of the aryl radical may be replaced by one or more groups such as saturated and/or unsaturated cyclic hydrocarbons which may be fused to the aromatic ring or to the aromatic rings or may be joined by a bond, or may be joined to one another via a suitable group. Suitable groups are those described above.

In the context of the present application, heteroaryl is understood to mean the aforementioned aryl compounds in which one or more carbon atoms of the radical are replaced by a heteroatom, e.g. N, O or S.

According to the present application, heterocyclo is understood to mean a saturated, partly unsaturated or unsaturated, cyclic radical in which one or more carbon atoms of the radical are replaced by a heteroatom such as N, O or S. Examples of heterocyclo radicals are piperazinyl, morpholinyl, tetrahydropyranyl, tetrahydrofuranyl, piperidinyl, pyrrolidinyl, oxazolinyl, pyridyl, pyrazyl, pyridazyl, pyrimidyl.

Substituted heterocyclo radicals are those heterocyclo radicals in which one or more hydrogen atoms bonded to one of the ring atoms are replaced by one or more groups such as halogen, alkyl, substituted alkyl, aryl, substituted aryl, heteroaryl, substituted heteroaryl, alkoxy, aryloxy, boryl, phosphino, amino, silyl, thio, seleno.

Alkoxy radicals are understood to mean radicals of the general formula —OZ¹ in which Z¹ is selected from alkyl, substituted alkyl, cycloalkyl, substituted cycloalkyl, heterocycloalkyl, substituted heterocycloalkyl and silyl. Suitable alkoxy radicals are, for example, methoxy, ethoxy, benzyloxy and t-butoxy.

The term aryloxy is understood to mean those radicals of the general formula —OZ² in which Z² is selected from aryl, substituted aryl, heteroaryl, substituted heteroaryl and combinations thereof. Suitable aryloxy radicals and heteroaryloxy radicals are phenoxy, substituted phenoxy, 2-pyridinoxy and 8-quinolinoxy.

Amino radicals are understood to mean radicals of the general formula —NZ³Z⁴ in which Z³ and Z⁴ are each independently selected from hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy and silyl.

Preferred aromatic and heteroaromatic hydrocarbons used in the amination process according to the invention are benzene, naphthalene, diphenylmethanes, anthracene, toluene, xylene, phenol and aniline, and also pyridine, pyrazine, pyridazine, pyrimidine and quinoline.

In a preferred embodiment, accordingly, at least one hydrocarbon from the group of benzene, naphthalene, diphenylmethanes, anthracene, toluene, xylene, phenol and aniline, and also pyridine, pyrazine, pyridazine, pyrimidine and quinoline is used.

It is also possible to use mixtures of the aromatic or heteroaromatic hydrocarbons mentioned. Particular preference is given to using at least one aromatic hydrocarbon from the group of benzene, naphthalene, anthracene, toluene, xylene, phenol and aniline, very particular preference to using benzene, toluene and naphthalene. Especially preferably, benzene is used in the process according to the invention.

An especially preferred aliphatic hydrocarbon for use in the process according to the invention is methane.

Aminating reagent

According to the invention, the reactant stream E comprises at least one aminating reagent. Suitable aminating reagents are those by which at least one amino group is introduced into the hydrocarbon used for direct amination. Examples of preferred aminating reagents are ammonia, primary and secondary amines, and compounds which release ammonia under the reaction conditions. It is also possible to use mixtures of two or more of the aforementioned aminating reagents.

An especially preferred aminating reagent is ammonia.

### Amino Hydrocarbons

In the process according to the invention, a reactant stream E comprising at least one hydrocarbon and at least one aminating reagent is converted to a reaction mixture R comprising at least one amino hydrocarbon and hydrogen. This affords at least one amino hydrocarbon which corresponds to the hydrocarbon used and comprises at least one amino group more than the hydrocarbon used. “Amino hydrocarbon” in the context of the present invention is accordingly understood to mean the reaction product of the hydrocarbons used in the process with the aminating reagent. This involves transferring at least one amino group from the aminating reagent to the hydrocarbon. In a preferred embodiment 1 to 6 amino groups, in a particularly preferred embodiment 1 to 3 amino groups, even more preferably 1 to 2 amino groups and especially preferably 1 amino group is/are transferred to the hydrocarbon. The number of amino groups transferred can be controlled through the molar ratio between aminating reagent and hydrocarbon to be aminated, and through the reaction temperature.

Typically, the ratio of aminating reagent to hydrocarbon is 0.5 to 9, preferably 1 to 5, more preferably 1.5 to 3.

In the case that the hydrocarbon used in the process according to the invention is benzene and the aminating reagent used is ammonia in a molar ratio in the range from 1 to 9, the amino hydrocarbon obtained is aniline.

In the case that the hydrocarbon used in the process according to the invention is toluene and the aminating reagent used is ammonia in a molar ratio in the range from 1 to 9, the amino hydrocarbon obtained is toluenediamine.

In the case that the hydrocarbon used in the process according to the invention is methane and the aminating reagent used is ammonia in a molar ratio in the range from 1 to 9, the amino hydrocarbon obtained is methylamine, dimethylamine or trimethylamine, or a mixture of two or more of the aforementioned amines.

In a particular embodiment, benzene is reacted with ammonia to give aniline. In a further particular embodiment, toluene is reacted with ammonia to give toluenediamine.

### Hydrogen Removal

In the process according to the invention, at least a portion of the hydrogen present in the reaction mixture R is electrochemically converted to protons by means of at least one membrane electrode assembly which is in contact with the reaction zone RZ on the retentate side, removed, and partly or fully reduced to hydrogen on the permeate side by applying a voltage to a cathode catalyst. Overall, the hydrogen is removed from the reaction zone and at least partly recovered. More particularly, the hydrogen is thus removed directly from the reaction zone and from the reaction mixture R.

The expression “applying a voltage” is understood to be synonymous with “generating a potential difference between anode and cathode”. Preferably, the hydrogen is removed at a potential difference of 0.05 to 2000 mV, preferably of 100 to 900 mV and more preferably of 100 to 800 mV. The application of the potential difference preferably increases the conversion of the hydrocarbons to amino hydrocarbons, since the hydrogen formed is removed electrochemically from the reaction zone.

The reaction mixture R typically comprises the coupling product of hydrocarbon(s) and aminating reagent(s) and hydrogen used. The reaction mixture R may additionally comprise unconverted reactants. The hydrogen is removed by means of a gas-tight membrane electrode assembly (MEA), the hydrogen to be removed being transported through the membrane in the form of protons.

The reaction mixture R is on the retentate side in contact with the MEA.

The electrochemical membrane of the MEA may, in the case of employment of temperatures less than 180° C., as known to those skilled in the art, be based on polymer materials (Nafion®, etc.) or phosphoric acid (Celtec, etc.). In addition, in the case of use of higher temperatures (of about 200 to about 800° C.), ceramic materials can be used.

The removal of the hydrogen by the process according to the invention is, depending on the membrane used (see below), performed preferably at temperatures of 20 to 800° C., especially of 50 to 700° C., more preferably of 70 to 350° C.

The removal of the hydrogen by the process according to the invention is preferably undertaken at pressures of 0.5 to 100 bar, preferably of 1 to 50 bar, especially at elevated pressure. In a preferred embodiment of the invention, the pressure difference between the retentate side and the permeate side of the membrane is below 1 bar, preferably below 0.5 bar; more preferably, there is no pressure difference.

According to the invention, at least a portion of the hydrogen in the reaction mixture R is removed electrochemically. Ideally, the removal is complete. For technical reasons, the membrane in many cases, however, cannot achieve complete removal, such that the reaction mixture R leaving the reaction zone still comprises small amounts of hydrogen. Preferably at least 30%, more preferably at least 50%, especially at least 70% and even more preferably at least 95%, especially at least 98%, of the hydrogen formed in the direct amination is removed.

### Electrode Catalysts

According to the invention, the material of which the anode consists can simultaneously also serve as the anode catalyst, and the material of which the cathode consists can simultaneously also serve as the cathode catalyst. However, it is also possible to use different materials in each case for the anode and the anode catalyst, or the cathode and the cathode catalyst.

In a preferred embodiment of the invention, the anode catalyst serves simultaneously as the amination catalyst. In this case, the anode catalyst preferably consists of at least one material of the amination catalysts mentioned below (under Gas diffusion layer). In the case of such a use, the hydrogen released in the reaction is removed directly from the catalyst surface of the amination catalyst by the proton-conducting membrane.

The electrodes can be produced using the customary materials known to those skilled in the art, for example Pt, Pd, Cu, Ni, Ru, Co, Cr, Fe, Mn, V, W, tungsten carbide, Mo, molybdenum carbide, Zr, Rh, Ag, Ir, Au, Re, Y, Nb, electrically conductive forms of carbon such as carbon black, graphite and nanotubes, and alloys and mixtures of the aforementioned elements.

The anode and the cathode may be produced from the same material or from different materials. Particularly preferred anode/cathode combinations are Pt/Pt, Pd/Pd, Pt/Pd, Pd/Pt, Pd/Cu, Pd/Ag, Ni/Pt, Ni/Ni, Cu/Cu and Fe/Fe, or alloys and mixtures of the aforementioned metals.

The anode catalyst and cathode catalyst may be selected from the same material or from different materials. The electrode catalyst materials used may be the customary compounds and elements which are known to those skilled in the art and which can catalyze the dissociation of molecular hydrogen to atomic hydrogen, the oxidation of hydrogen to protons and the reduction of protons to hydrogen. Suitable materials for this purpose include Pd, Pt, Cu, Ni, Ru, Fe, Co, Cr, Mn, V, W, tungsten carbide, Mo, molybdenum carbide, Zr, Rh, Ag, Ir, Au, Re, Y, Nb, and alloys and mixtures thereof, preference being given in accordance with the invention to Pd, Pt, Ni and Cu. The elements and compounds mentioned above as electrode catalyst material may also be present in supported form, preference being given to using carbon as the support.

The anode catalyst used on the retentate side may simultaneously serve as the catalyst for the conversion of hydrocarbon to amino hydrocarbon (amination catalyst). In a further preferred embodiment, the amination catalyst is placed directly on the electrode catalyst. In this case too, the hydrogen released in the reaction is removed directly from the catalyst surface of the amination catalyst by the proton-conducting membrane.

### Gas Diffusion Layer (GDL)

In order to ensure good contact of the membrane with the hydrogen present on the retentate side and good transport of the hydrogen removed away on the permeate side, the anode layers and/or cathode layers are preferably contacted with gas diffusion layers (GDLs). The GDLs are preferably in the form of plates with a grid-like surface structure composed of a system of fine channels, or they are layers of porous material such as nonwoven fabric, woven fabric or paper. The combination of GDL and electrode layer is generally referred to as gas diffusion electrode (GDE). The GDL conducts the hydrogen to be removed close to the membrane and the anode catalyst on the retentate side, and facilitates the transport of the hydrogen formed away on the permeate side.

In a further preferred embodiment, the GDL includes the amination catalyst. In this embodiment, the advantages of a GDL and the aforementioned advantages of a reaction zone in contact with the MEA can be combined particularly efficiently. If the modified GDL in the abovementioned embodiment serves as a catalyst for the conversion of hydrocarbon to amino hydrocarbon (amination catalyst), the hydrogen released in the reaction can be removed directly from the catalyst surface of the amination catalyst by the proton-conducting membrane.

Suitable catalysts are in principle all known amination catalysts. These can be applied to the GDL, for example, by means of impregnation, precipitation, coating or similar processes. The catalysts used may, for example, be metal catalysts based on nickel, iron, cobalt, copper, noble metals (NM) or alloys of these metals mentioned. Preferred NMs are Ru, Rh, Pd, Ag, Ir, Pt and Au. In a particular embodiment, the NMs are not used alone, but in an alloy with one or more other transition metals, such as Co, Cu, Fe and nickel. Examples of suitable catalysts are NiNM, CuNM, NiCuNM; CoCuNM; NiCoCuNM, NiMoNM, NiCrNM, NiReNM, CoMoNM, CoCrNM, CoReNM, FeCuNM, FeCoCuNM, FeMoNM, FeReNM alloys. NM here is preferably Pt, Pd, Ag, Ir, especially preferably Ag and/or Ir. Especial preference is additionally given to NiCuNM where NM is selected from Pt, Pd, Ag and/or Ir.

The above-described GDL comprising amination catalyst may be installed either upstream (directly on the membrane) or downstream of the electrode catalyst.

Regeneration of the active components present on the GDL is possible by methods known to those skilled in the art. This means that the regeneration can be conducted, for example, in a reductive or oxidative atmosphere. In a preferred embodiment, the regeneration is conducted reductively.

### Membranes

The membrane is preferably in the form of a plate or of a tube, it being possible to use the customary membrane arrangements known from the prior art for separation of gas mixtures, for example shell-and-tube or insertable plate membrane.

The MEA used in accordance with the invention is gas-tight, which means that it has at most low permeability to gases. Such permeabilities of gases arise particularly through porosity, through which gases pass in atomic or molecular form from one side to the other side of the MEA, or by mechanisms through which gases can be transported unselectively through the MEA, for example by adsorption, dissolution in the membrane, diffusion and desorption. The imperviosity of the membrane electrode assembly (MEA) can be ensured by a gas-tight membrane, by a gas-tight electrode, or a gas-tight electrode catalyst, or else by a combination thereof. A gas-tight MEA has, more particularly, a density measured by the Archimedes method of more than 90%, preferably more than 95% and more preferably more than 98% of the bulk density. In this case, it is ensured that the MEA is gas-tight. In addition, the membrane used in accordance with the invention selectively conducts protons, which means more particularly that it is not electron-conductive, and that it is permeable exclusively to protons in relation to the reaction mixture R.

In principle, useful materials for the membranes include all materials which are known to those skilled in the art and from which selectively proton-conducting membranes can be formed. These include, for example, the materials described in the following documents: WO 2011/003932; WO 2011/003933; WO 2011/003934; WO 2011/003964; Int. J. Hydr. Energy 2010, 35, 9349; J. Pow. Sourc. 2008, 180,15; J. Pow. Sourc. 2008, 179, 92; J. Pow. Sourc. 2008, 176,122; Electrochem. Commun. 2008, 10, 1005; Ionics 2006, 12, 103; Annu. Rev. Mater. Res. 2003, 33, 333; Solid State Ion. 1999, 125, 271.

At temperatures less than 200° C., preference is given to using polymer membranes. Particularly suitable for this purpose are the following polymers: sulfonated polyetheretherketones (S-PEEK), sulfonated polybenzimidazoles (S-PBI) and sulfonated hydrofluorocarbon polymers (NAFION®). In addition, it is possible to use perfluorinated polysulfonic acids, styrene-based polymers, poly(arylene ethers), polyimides and polyphosphazenes. In addition, it is possible to use phosphoric acid-based membranes (PAFCs): those based on polybenzimidazole and phosphoric acid are sold, for example, under the Celtec-P® name by BASF SE; but it is also possible to use other polymers such as Teflon, alone or in combination with other inorganic substances, for example SiC, as the matrix.

At temperatures greater than 200° C., preference is given to using ceramic membranes. In principle, all proton-conducting ceramics and inorganic materials known to those skilled in the art are useful: the following are especially suitable:

Ceramic membranes composed of heteropolyacids, for example H₃Sb₃B₂O₁₄.10H₂O, H₂Ti₄O₉. 12H₂O and HSbP₂O₈.10H₂O; acidic zirconium silicates, phosphates and phosphonates in layer structure, such as K₂ZrSi₃O₉, K₂ZrSi₃O₉, alpha-Zr(HPO₄)₂.nH₂O, gamma-Zr(PO₄)—(H₂PO₄) .2H₂O, alpha- and gamma-Zr sulfophenylphosphonate or sulfoarylphosphonate; non-layered oxide hydrates such as antimony acid (Sb₂O₅.2H₂O), V₂O₅.nH₂O, ZrO₂.nH₂O, SnO₂.nH₂O and Ce(HPO₄)₂.nH₂O. In addition, it is possible to use oxo acids and salts which comprise, for example, sulfate, selenate, phosphate, arsenate, nitrate groups, etc. Particularly suitable are oxo anion systems based on phosphates or complex heteropolyacids such as polyphosphate glasses, aluminum polyphosphate, ammonium polyphosphate and polyphosphate compositions such as NH₄PO₃/(NH₄)₂SiP₄O₁₃ and NH₄PO₃/TiP₂O₇. It is additionally possible to use oxidic materials such as brownmillerite, fluorite, and phosphates with apatite structure, pyrochlore minerals and especially perovskites. In general, it is possible to use all proton-conducting materials, for example including zeolites, aluminosilicates, xAl₂O₃(1−x)SiO₂, SnP₂O₇, Sn₁₋ₓInₓP₂O₇ (X=0.0−0.2), oxides treated with inorganic acids (SiO₂, Al₂O₃, TiO₂, ZrO₂), etc.

Perovskites have the basic formula AB₁₋ₓMₓO_{3-y}, where M is a trivalent rare earth element which serves for doping, and y denotes the oxygen deficiency in the perovskite oxide lattice. A may be selected, for example, from Mg, Ca, Sr and Ba. B may be selected, inter alia, from Ce, Zr and Ti. For A, B and M, it is also possible to independently select different elements from the respective groups.

In addition, it is possible to use structurally modified glasses, such as chalcogenide glasses, PbO—SiO₂, BaO—SiO₂ and CaO—SiO₂.

Further proton-conducting ceramics and oxides are SrCeO₃, BaCeO₃, Yb:SrCeO₃, Nd:BaCeO₃, Gd:BaCeO₃, Sm:BaCeO₃, BaCaNdO₉, Y:BaCeO₃, Y:BaZrCeO₃, Pr-doped Y:BaCeO₃, Gd:BaCeO₃, BaCe_{0.9}Y_{0.1}O_{2.95} (BYC), SrCe_{0.95}Yb_{0.05}O_{3-α}, BaCe_{0.9}Nd_{0.10}O_{3-α}, CaZr_{0.96}In_{0.04}O_{3-α} (αdenotes the number of oxygen defect sites per formula unit of the oxide of the perovskite type); Sr-doped La₃P₃O₉, Sr-doped LaPO₄, BaCe_{0.9}Y_{0.1}O_{3-α} (BCY), BaZr_{0.9}Y_{0.1}O_{3-α} (BZY), Ba₃Ca_{1.18}Nb_{1.82}O_{8.73} (BCN18), (La_{1.95}Ca_{0.05})Zr₂O_{7-α}, La₂Ce₂O₇, Eu₂Zr₂O₇, H₂S/(B₂S₃ or Ga₂S₃)/GeS₂, SiS₂, As₂S₃ or Csl; BaCe_{0.8}Gd_{0.2}O_{3-α} (BCGO); Gd-doped BaCeO₃ such as BaCe_{0.85}Y_{0.15}O_{3-α} (BCY15) and BaCe_{0.8}Sm_{0.2}O_{3-α}, xAl₂O₃ (1−X)SiO₂, SnP₂O₇, Sn₁₋ₓInₓP₂O₇ (x=0.0−0.2).

The materials used for the selectively proton-conducting membrane are preferably the aforementioned ceramic membranes.

In the case of use of polymer membranes, these are typically moistened by the presence of about 0.5 to 30% by volume of water on at least one side of the membrane.

### Conditions of the Direct Amination

Processes for direct amination of hydrocarbons to give amino hydrocarbons comprising the reaction of a reactant stream E comprising at least one hydrocarbon and at least one aminating reagent to give a reaction mixture R comprising at least one amino hydrocarbon and hydrogen (referred to hereinafter in abbreviated form as direct amination(s)) in a reaction zone RZ are known to those skilled in the art. With regard to the reaction conditions of the known direct aminations, there is no fundamental restriction in the context of the present invention. The direct amination can be performed under oxidative or nonoxidative conditions. The direct amination can additionally be performed under catalytic or noncatalytic conditions.

The direct amination preferably takes place in the presence of a catalyst. The direct amination preferably takes place under nonoxidative conditions.

Nonoxidative means, in relation to the direct amination, that the concentration of oxidizing agents such as oxygen or nitrogen oxides in the reactants used (reactant stream E) is below 5% by weight, preferably below 1% by weight, more preferably below 0.1% by weight (based in each case on the total weight of the reactant stream E).

The reactant stream E is most preferably free of oxygen. Particular preference is likewise given to a concentration of oxidizing agent in the reactant stream E equal to or less than the concentration of oxidizing agents in the source from which the hydrocarbons and aminating reagents used originate.

The reaction conditions in the direct aminations depend upon factors including the hydrocarbon to be aminated and the catalyst used.

The direct amination is effected generally at temperatures of 20 to 800° C., preferably 50 to 700° C., more preferably 70 to 350° C.

The reaction pressure in the direct amination is preferably 0.5 to 100 bar, preferably 1 to 50 bar, particularly at elevated pressure.

The residence time in the case of a batchwise process regime for the direct amination is generally 15 minutes to 24 hours, preferably 15 minutes to 8 hours, more preferably 15 minutes to 4 hours. In the case of performance in a continuous process, the residence time is generally 0.1 second to 20 minutes, preferably 0.5 second to 20 minutes. For the preferred continuous direct aminations, “residence time” in this context means the residence time of the reactant stream E in the reaction zone.

Like the reaction conditions, the relative amount of the hydrocarbon used and of the aminating reagent depends on the amination reaction conducted. In general, at least stoichiometric amounts of hydrocarbon and aminating reagent are used. Preference is given to using one of the reactants in a stoichiometric excess in order to achieve an equilibrium shift to the side of the desired product and hence a higher conversion. Preference is given to using the aminating reagent in a stoichiometric excess in relation to the hydrocarbon. The molar ratio of aminating reagent to hydrocarbon is 0.5 to 9, preferably 1 to 5, more preferably 1.5 to 3.

The reactor types suitable in relation to process step (a) in the process according to the invention are not restricted in principle and are known per se to the person skilled in the art. The invention requires, however, that the retentate side of the MEA is in contact with the reaction zone. Accordingly, the reactor and the MEA form one unit. The reactors can each be used as a single reactor, as a series of single reactors and/or in the form of two or more parallel reactors. The process according to the invention can be performed as a batchwise, semibatchwise or continuous reaction. The specific reactor construction and the performance of the reaction may vary depending on the amination process to be performed, the state of matter of the aromatic hydrocarbon to be aminated, the reaction times required and the nature of the catalyst used. Preference is given to performing the process according to the invention for direct amination in a pressure-stable electrochemical cell.

### Workup of the Product Stream

After the removal of the hydrogen from the reaction mixture R by means of at least one MEA, a product stream P is obtained. This comprises at least one amino hydrocarbon and possibly unconverted reactants, such as hydrocarbons and aminating reagents, and possibly unremoved hydrogen which thus remains in the product stream P. In a preferred embodiment, the product stream P comprises less than 500, preferably less than 200 and especially preferably less than 100 ppm of hydrogen.

Optionally, the hydrogen remaining in the product stream P can be removed by contacting the product stream P with one or more MEAs again in a downstream step. In a preferred embodiment, however, the hydrogen is removed completely or virtually completely from the reaction mixture, such that a downstream removal of hydrogen from the product stream P can be dispensed with.

In one process variant (variant A), the amino hydrocarbon and the aminating reagent are removed from the product stream P, the sequence of removal being freely selectable. Preference is given, however, to first removing the aminating reagent, then the amino hydrocarbon. The worked-up stream S1 thus obtained comprises the unconverted hydrocarbon, which, in a preferred embodiment, can be used again in the direct amination. For this purpose, the hydrocarbon from stream S1 is either added to the reactant stream E or is recycled directly to the reaction zone. Amino hydrocarbon and aminating reagent can be removed by commonly known methods familiar to the person skilled in the art, for example by condensation, distillation or extraction. The choice of the temperature and pressure range is guided by the physical properties of the compounds to be separated and is known to those skilled in the art.

For instance, the product stream P can be cooled to 50° C. to 250° C., preferably to 70° C. to 200° C., more preferably to 80° C. to 150° C., at pressures in the range from 0 to 5 bar, preferably 0.5 to 2 bar, more preferably 0.8 to 1.5 bar and especially at standard pressure. In the course of this, the amino hydrocarbons generally condense, while unconverted hydrocarbon and aminating reagent and any hydrogen still present remain in gaseous form and can thus be removed by customary methods, for example with a gas-liquid separator.

The liquid constituents thus obtained comprise the amino hydrocarbon and unconverted hydrocarbon; the amino hydrocarbon and hydrocarbon are likewise removed by methods known to those skilled in the art, such as distillation, rectification or acid extraction.

The amounts of hydrocarbon and aminating reagent supplied to the worked-up stream S1 are chosen so as to comply with the molar ratios of hydrocarbon and aminating reagent required for the direct amination.

In the case of direct amination of benzene and ammonia to give aniline and hydrogen, the product stream P comprises essentially aniline, unconverted benzene and ammonia, and possibly by-products and residues of hydrogen. In one embodiment, the product stream P is first separated by condensation into a gaseous phase comprising ammonia and any residues of hydrogen, and a liquid phase comprising aniline and benzene. The liquid phase is subsequently separated into aniline and benzene by distillation, rectification or acid extraction. The benzene (stream S1) is reused in the direct amination. The aniline thus obtained can optionally be subjected to further workup steps.

In a further process variant (variant B), the product stream P is reused in the direct amination. This can be achieved either through a cycle gas stream or series-connected reactors, for which purpose the product stream P can be added to the reactant stream E or introduced separately and directly into the reaction zone RZ. In a preferred embodiment, the product stream P is conducted into a reaction zone RZ until a concentration of amino hydrocarbon has accumulated in the product stream P which enables economically viable workup. For this purpose, preference is given to recycling the product stream P to the reaction zone RZ one to twenty times, preferably one to ten times, more preferably one to five times and especially preferably one to three times. In this mode of operation, the amounts of hydrocarbon and aminating reagent supplied to the recycled product stream P are chosen so as to comply with the molar ratios of hydrocarbon and aminating reagent required for the direct amination.

The amino hydrocarbon is worked up and removed by the process described under variant A.

In a further process variant (variant C), the amino hydrocarbon is removed from the product stream P. The worked-up stream S2 thus obtained comprises, in this process variant, unconverted hydrocarbon and aminating reagent. This worked-up stream S2 can be reused in the direct amination. The worked-up product stream can, as described in variant B, for this purpose be added to the reactant stream E or introduced directly into a reaction zone RZ. The amino hydrocarbon is removed by methods known to those skilled in the art, for example by condensation, distillation or acid extraction. In variant C, preference is given to removing the amino hydrocarbon from the product stream P by acid extraction. The amounts of hydrocarbon and aminating reagent supplied to the recycled product stream P are selected so as to comply with the molar ratios of hydrocarbon and aminating reagent required for the direct amination.

The present invention can advantageously be applied to all reactions in which hydrogen is formed.

The present invention accordingly provides a process for continuously removing the hydrogen formed in a reaction, wherein the hydrogen formed in a reaction is at least partly electrochemically removed by means of a gas-tight membrane electrode assembly (MEA) which is in contact with the reaction zone RZ on the retentate side and which has at least one selectively proton-conducting membrane, at least a portion of the hydrogen being oxidized over the anode catalyst to protons on the retentate side of the membrane, and the protons, after passing through the membrane, being partly or fully reduced by applying a voltage over the cathode catalyst to give hydrogen on the permeate side.

The reaction is especially an equilibrium reaction. This allows a reaction equilibrium to be shifted in a desired direction.

The present invention further provides a process for removing hydrogen from a gas mixture comprising hydrogen and at least one organic compound, wherein the hydrogen present in the gas mixture is at least partly electrochemically removed by means of a gas-tight membrane electrode assembly (MEA) which is in contact with the reaction zone RZ on the retentate side and which has at least one selectively proton-conducting membrane, at least a portion of the hydrogen being oxidized over the anode catalyst to protons on the retentate side of the membrane, and the protons, after passing through the membrane, being partly or fully reduced by applying a voltage over the cathode catalyst to give hydrogen on the permeate side.

The invention is illustrated by the examples which follow, without restricting it thereto.

### EXAMPLE 1

Direct amination of benzene with in situ removal of H₂ directly from the reaction zone, Pd foil as anode catalyst and amination catalyst

An electrochemical cell was charged with benzene preheated at a temperature of 200° C. (0.4 ml/h) and ammonia (17 I (STP)/h) (reactant stream E). The cell comprised a gas-tight MEA with sufficient active area (45 cm²). The proton-conducting membrane used in the MEA was a phosphoric acid-impregnated polybenzimidazole (PBI) membrane. The anode electrode used in the MEA, and at the same time the amination catalyst, was a palladium foil (manufacturer: Goodfellow, thickness 10 μm); the cathode used was an ELAT gas diffusion electrode with Pt loading of 1 mg/cm² (manufacturer: BASF Fuel Cell GmbH). The Pd foil was gas-tight.

The MEA used accordingly had a layer structure with—beginning on the retentate side—the following layer sequence:
1. gas diffusion layer without catalyst,
2. palladium foil (manufacturer: Goodfellow, thickness 10 μm),
3. ELAT gas diffusion electrode with Pt loading 1 mg/cm²,
4. proton-conducting membrane (H₃PO₄/PBI) and
5. ELAT gas diffusion electrode with Pt loading 1 mg/cm².

The reactant stream comprised benzene, ammonia and nitrogen and was introduced into the electrochemical cell to the reaction zone on the anode side. On the retentate side, the hydrogen was oxidized over the anode catalyst to protons, which passed through the membrane and were reduced by applying a voltage over the cathode catalyst to give hydrogen on the permeate side. The potential difference applied was 200 mV. The reactor output obtained on the anode side was a mixture of benzene, aniline, hydrogen, nitrogen and ammonia. A majority (>90% by volume) of the hydrogen was removed electrochemically from the reaction zone.

### EXAMPLE 2

Direct amination of benzene with in situ removal of H₂ directly from the reaction zone, Pd foil as anode catalyst, gas diffusion layer comprising NiCu alloy as amination catalyst

An electrochemical cell was charged with benzene preheated at a temperature of 200° C. (0.4 ml_{liquid}/h) and ammonia (17I (STP)/h) (reactant stream E). The cell comprised a gas-tight MEA with sufficiently active area (45 cm²). The proton-conducting membrane used in the MEA was a phosphoric acid-impregnated polybenzimidazole (PBI) membrane. The anode electrode used in the MEA was a palladium foil (manufacturer: Goodfellow, thickness 10 μm); the cathode used was an ELAT gas diffusion electrode with a Pt loading of 1 mg/cm² (manufacturer: BASF Fuel Cell GmbH). The advantage of Pd foil is that it is gas-tight and hence offers protection of the membrane from ammonia. In addition, a gas diffusion layer (GDL) was installed on the retentate side of the electrochemical cell, which consists of nonwoven carbon fabric comprising amination catalyst (NiCu alloy). This GDL was applied directly to the Pd foil.

The MEA accordingly had a layer structure with—beginning on the retentate side—the following layer sequence:
1. GDL comprising amination catalyst, consisting of a carbon fabric comprising finely dispersed NiCu alloy,
2. palladium foil (manufacturer: Goodfellow, thickness 10 μm),
3. ELAT gas diffusion electrode with Pt loading 1 mg/cm²,
4. proton-conducting membrane (H₃PO₄/PBI) and
5. ELAT gas diffusion electrode with Pt loading 1 mg/cm².

The reactant stream comprised benzene, ammonia and nitrogen, and was introduced into the electrochemical cell to the reaction zone on the anode side. On the retentate side, the hydrogen was oxidized over the anode catalyst to protons, which passed through the membrane and reduced by applying a voltage over the cathode catalyst to give hydrogen on the permeate side. The potential difference applied was 200 mV.

The reactor output obtained on the anode side was a mixture of benzene, aniline, hydrogen, nitrogen and ammonia. A majority (>90% by volume) of the hydrogen was removed from the reaction mixture R directly from the reaction zone by means of the electrochemical membrane. Thus, the equilibrium conversion of benzene to aniline was increased.

### EXAMPLE 3

The GDLs comprising amination catalysts mentioned in Example 2 are obtained as follows:

Alternative 1:
1. A carbon fabric (from SGL Carbon or Freudenberg) is soaked with an alcoholic solution of Ni salts and Cu salts.
2. The fabric thus obtained is dried at elevated temperature under reduced pressure and then calcined.
3. The GDL now comprising nickel oxide and copper oxide is activated in a hydrogen stream and then passivated with air.

Alternative 2: Preparation by spraying of catalyst suspension

Alternative 3: Preparation by screen printing of catalyst paste

### EXAMPLE 4

If the direct amination of hydrocarbons is performed at elevated temperatures, it is necessary to use proton-conducting ceramic membranes for hydrogen removal. This purpose may be served by di- or polyphosphates which are obtained as follows:

Sn_{0.9}In_{0.1}P₂O₇:

The oxides of the diphosphate to be prepared (SnO₂ and In₂O₃) are slurried and homogenized in the given amounts in an excess of phosphoric acid. The mixture is subsequently dried and calcined at >600° C. for two hours. The product thus obtained exhibits a conductivity of ˜1 mS/cm as a membrane at 300° C. under water-moist conditions.

### EXAMPLE 5

Perovskite materials are suitable as proton-conducting ceramic membranes for hydrogen removal:

BaCe_{0.45}Zr_{0.45}In_{0.1}O_{3-d}:

BaCO3, CeO2, ZrO2 and In2O3 are mixed in the given stoichiometric amounts, moistened and homogenized in a ball mill. This is followed by drying at 120° C. overnight and calcination of the resulting powder at 1250° C. Thereafter, the powder is ground in a ball mill to give particles <5 pm and processed to give the membrane. An optional calcination step (T>1000° C.) may follow in order to ensure the gas-tightness of the membrane.

1. A process for direct amination of hydrocarbons to amino hydrocarbons, comprising
(a) the reaction of a reactant stream E comprising at least one hydrocarbon and at least one aminating reagent to give a reaction mixture R comprising at least one amino hydrocarbon and hydrogen in a reaction zone RZ, and
(b) electrochemical removal of at least a portion of the hydrogen formed in the reaction from the reaction mixture R by means of at least one gas-tight membrane electrode assembly which is in contact with the reaction zone RZ on the retentate side and which has at least one selectively proton-conducting membrane,
at least a portion of the hydrogen being oxidized over an anode catalyst to protons on the retentate side of the membrane, and the protons, after passing through the membrane, being partly or fully reduced by applying a voltage over a cathode catalyst to give hydrogen on the permeate side.

2. The process according to claim 1, wherein the hydrogen is removed at a potential difference of 0.05 to 2000 mV.

3. The process according to claim 2, wherein the application of the potential difference increases the conversion of the reaction of the hydrocarbons to amino hydrocarbons.

4. The process according to claim 1, wherein the hydrogen is removed from the reaction zone RZ.

5. The process according to claim 1, wherein the electrode catalyst present on the retentate side serves simultaneously as an amination catalyst for the conversion of the hydrocarbons to amino hydrocarbons.

6. The process according to one claim 1, wherein a gas distributor layer present on the retentate side has been functionalized with an amination catalyst.

7. The process according to claim 1, wherein the electrodes of the membrane electrode assembly (MEA) comprising an amination catalyst serve for the conversion of the hydrocarbons to amino hydrocarbons.

8. The process according to claim 1, wherein, in the removal of hydrogen, the pressure difference between the retentate side and the permeate side of the membrane is below 1 bar.

9. The process according to claim 1, wherein the electrodes of the membrane electrode assembly are configured as gas diffusion electrodes.

10. The process according to claim 1, wherein the electrodes of the membrane electrode assembly are configured as metallic foils.

11. The process according to claim 1, wherein the selectively proton-conducting membrane used comprises membranes selected from the group of ceramic membranes, polymer membranes or phosphoric acid-based membranes.

12. The process according to claim 1, wherein removal of at least a portion of the hydrogen formed affords a product stream P which is reused in the direct amination.

13. The process according to claim 1, wherein removal of at least a portion of the hydrogen formed affords a product stream P from which the amino hydrocarbon and optionally the aminating reagent are removed to form a worked-up stream S1 which is reused in the direct amination.

14. The process according to claim 1, wherein the hydrocarbon used is methane and the aminating reagent ammonia, and the amino hydrocarbon formed is at least one amine from the group of methylamine, dimethylamine and trimethylamine.

15. The process according to claim 1, wherein the hydrocarbon used is benzene and the aminating reagent ammonia, and the amino hydrocarbon formed is at least one amine from the group of aniline, diaminobenzene and triaminobenzene.

16. The process according to claim 1, wherein the hydrocarbon used is toluene and the aminating reagent ammonia, and the amino hydrocarbon formed is at least one amine from the group of tolueneamine, toluenediamine and toluenetriamine.

17. The process according to claim 1, wherein the working pressure in the reaction zone is from 0.5 to 100 bar.

18. A process for continuously removing the hydrogen formed in a chemical reaction, wherein the hydrogen formed in a reaction in a reaction zone RZ is at least partly electrochemically removed from the reaction zone RZ by means of a gas-tight membrane electrode assembly (MEA) which is in contact with the reaction zone RZ on the retentate side and which has at least one selectively proton-conducting membrane, at least a portion of the hydrogen being oxidized over the anode catalyst to protons on the retentate side of the membrane, and the protons, after passing through the membrane, being partly or fully reduced by applying a voltage over the cathode catalyst to give hydrogen on the permeate side.

19. The process according to claim 18, wherein the reaction is an equilibrium reaction.

20. A process for removing hydrogen from a gas mixture comprising hydrogen and at least one organic compound, wherein the hydrogen present in the gas mixture is at least partly electrochemically removed by means of a gas-tight membrane electrode assembly (MEA) having at least one selectively proton-conducting membrane, at least a portion of the hydrogen being oxidized over the anode catalyst to protons on the retentate side of the membrane, and the protons, after passing through the membrane, being partly or fully reduced by applying a voltage over the cathode catalyst to give hydrogen on the permeate side.
